(19) United States
(12) Patent Application Publication
(10) Pub. No.: US 2011/0112410 A1
(43) Pub. Date: May 12, 2011

(54) OPTICAL PROBE, DRIVE CONTROL METHOD THEREFOR, AND ENDOSCOPE APPARATUS

(76) Inventor: Kazuhiro HIROTA, Ashigarakami-gun (JP)

(21) Appl. No.: 12/942,744

(22) Filed: Nov. 9, 2010

(30) Foreign Application Priority Data
Nov. 10, 2009 (JP) NO. 2009-257277

(51) Int. Cl. A61B 1/07; A61B 6/00

(52) U.S. Cl. 600/478; 600/115; 600/104

(57) ABSTRACT

An optical probe, comprising:
a sheath to be inserted into a body cavity;
an optical fiber inside the sheath;
an optical component attached to a distal end portion of the optical fiber inside the sheath;
wherein a light beam transmitted through the optical fiber is emitted from the optical component toward a body tissue in the body cavity, and
wherein the sheath comprises
a plurality of balloons spaced at predetermined intervals on a part on the distal end portion side of an outer periphery and capable of inflating/deflating in a radial direction orthogonal to a longitudinal axis,
a suction inlet located between the plurality of balloons to draw the body tissue at the part on the distal end portion side of the outer periphery by suction,
a balloon inflation port connected to the balloons, and
a suction port connected to the suction inlet, and
the plurality of balloons are airtightly locked to an body cavity inner wall by supplying a fluid or a gas through the balloon inflation port to pressurize and inflate the balloons, and pressure in a space formed between the plurality of locked balloons and the body cavity inner wall is reduced through the suction port to cause the body tissue to adhere strongly to the part on the distal end portion side of the outer periphery by suction.

### BACKGROUND OF THE INVENTION

1. Field of the Invention

The present invention relates to an optical probe for acquiring an optical coherent tomographic image of the interior of a body cavity, a drive control method for the optical probe, and an endoscope apparatus and, more particularly, to an optical probe which stably transmits and receives a measuring light beam in a lumen, a drive control method for the optical probe, and an endoscope apparatus.

2. Description of the Related Art

Conventionally, diagnostic imaging that includes rendering a tomographic image of a living body by inserting an optical probe into a body cavity of a blood vessel, a bile duct, a pancreatic duct, a stomach, an esophagus, or the large intestine and performing radial scanning has been widely performed. As an example, an optical coherent tomography (OCT) apparatus which, when a probe which incorporates an optical fiber having an optical lens and an optical mirror attached to its distal end is inserted in a body cavity, emits a light beam into the body cavity while performing radial scanning using the optical mirror arranged on the distal end side of the optical fiber and renders a cross-sectional image of the body cavity on the basis of a light beam reflected from a tissue is used.

Excellent features of OCT include the capability to render a tomographic image with a resolution of 10 μm. In order to take advantage of the feature, fluctuations in the positional relationship with a target body tissue need to be prevented. As shown in FIG. 14, a sheath 901 of an OCT probe 900 is brought into contact with a lumen inner wall 902, and a measuring light beam is applied from a ball lens 904 which is provided at the distal end of a shaft 903 incorporating a fiber (not shown) by radial scanning (or spiral scanning).

However, when the interior of a body cavity is observed by OCT, the positional relationship between the OCT probe 900 and a body tissue varies during the observation due to beating or pulsation of the body tissue, movement of the hands of an operator, or the like, the above-described feature of OCT cannot be taken advantage of. Accordingly, there is strong demand for fixation of the relative positional relationship between the OCT probe 900 and a body tissue.

To meet this demand, an OCT system in which a balloon is provided at the distal end of an OCT probe, and the OCT probe is fixed to a body tissue by inflation of the balloon is proposed (Japanese Patent Application Laid-Open No. 2007-75403).

However, the OCT system disclosed in Japanese Patent Application Laid-Open No. 2007-75403, which fixes the OCT probe to a body cavity by inflating the balloon, suffers from the following problems:

1) a part to be observed is away from the center of the probe and has an unobservable area; and
2) the spacing between scan lines for a part away from the center is large in radial scanning, and the quality of an image of the part is low.

More specifically, the above-described OCT system of Japanese Patent Application Laid-Open No. 2007-75403, which fixes the OCT probe to a body cavity by inflating the balloon, cannot display an image of a part away from the OCT probe because OCT, in principle, has a shallow displayable depth equal to a coherence length of about 3 mm. Additionally, since a measuring light beam is radially emitted in radial scanning, the spacing between scan lines for a part away from the center is large, and the quality of an image of the part is low.

### SUMMARY OF THE INVENTION

The present invention has been made in consideration of the circumstances, and has as its object to provide an optical probe capable of stably locking an OCT probe in a body cavity and acquiring a blur-free tomographic image with high resolution, a drive control method for the optical probe, and an endoscope apparatus.

In order to achieve the above-described object, an optical probe according to a first aspect of the present invention is an optical probe, comprising:

a sheath to be inserted into a body cavity;

an optical fiber inside the sheath;

an optical component attached to a distal end portion of the optical fiber inside the sheath;

wherein a light beam transmitted through the optical fiber is emitted from the optical component toward a body tissue in the body cavity, and

wherein the sheath comprises a plurality of balloons spaced at predetermined intervals on a part on the distal end portion side of an outer periphery and capable of inflating/deflating in a radial direction orthogonal to a longitudinal axis,

a suction inlet located between the plurality of balloons to draw the body tissue at the part on the distal end portion side of the outer periphery by suction,

a balloon inflation port connected to the balloons, and

a suction port connected to the suction inlet, and

the plurality of balloons are airtightly locked to an body cavity inner wall by supplying a fluid or a gas through the balloon inflation port to pressurize and inflate the balloons, and pressure in a space formed between the plurality of locked balloons and the body cavity inner wall is reduced through the suction port to cause the body tissue to adhere strongly to the part on the distal end portion side of the outer periphery by suction.

In the optical probe according to the first aspect, the plurality of balloons are airtightly locked to the body cavity inner wall by supplying the fluid through the balloon inflation port to pressurize and inflate the balloons, and the pressure in the space formed between the plurality of locked balloons and the body cavity inner wall is reduced through the suction port to cause the body tissue to adhere strongly to the part on the distal end portion side of the outer periphery by suction. This configuration makes it possible to stably lock an OCT probe in a body cavity and acquire a blur-free tomographic image with high resolution.

As in the case of an optical probe according to a second aspect of the present invention, the optical probe according to the first aspect is preferably configured such that the balloon inflation port and the suction port are provided at a proximal end portion of the sheath.

As in the case of an optical probe according to a third aspect of the present invention, the optical probe according to one of the first and second aspects is preferably configured such that the optical fiber is arranged in a drive shaft which rotatably drives the optical fiber, and radial scanning is performed in the body cavity by rotatably driving the optical component.

As in the case of an optical probe according to a fourth aspect of the present invention, the optical probe according to the third aspect is preferably configured such that the drive shaft is also movable in a longitudinal direction, and spiral scanning is performed in the body cavity by rotatably driving the optical component and driving the optical component back and forth within a longitudinal driving range.

As in the case of an optical probe according to a fifth aspect of the present invention, the optical probe according to any one of the first to fourth aspects is preferably configured such that the optical component comprises a ball lens having a reflecting surface which bends a traveling direction of the light beam transmitted through the optical fiber almost at a right angle.

As in the case of an optical probe according to a sixth aspect of the present invention, the optical probe according to any one of the first to fifth aspects is preferably configured such that the optical fiber transmits wavelength swept laser light into the body cavity.

As in the case of an optical probe according to a seventh aspect of the present invention, the optical probe according to any one of the first to sixth aspects is preferably configured such that the balloons are each thicker at two end portions than at a central portion.

As in the case of an optical probe according to an eighth aspect of the present invention, the optical probe according to any one of the first to seventh aspects preferably further comprises a fluid supply control device which detects internal pressure of each of the balloons and controls supply of the fluid through the balloon inflation port in order to keep the balloon airtightly locked to the body cavity inner wall.

As in the case of an optical probe according to a ninth aspect of the present invention, the optical probe according to any one of the first to eighth aspects is preferably configured such that the fluid is one of an X-ray contrast medium and a fluid containing an X-ray contrast medium.

As in the case of an optical probe according to a tenth aspect of the present invention, the optical probe according to any one of the first to eighth aspects is preferably configured such that the fluid is saline.

As in the case of an optical probe according to an 11th aspect of the present invention, the optical probe according to any one of the first to tenth aspects is preferably configured such that the optical fiber transmits wavelength swept laser light into the body cavity.

A drive control method for an optical prove according to a 12th aspect of the present invention is a drive control method for an optical probe which comprises an optical fiber and an optical component attached to a distal end portion of the optical fiber inside a sheath to be inserted into a body cavity and emits a light beam transmitted through the optical fiber from the optical component toward a body tissue in the body cavity, the optical probe further comprising a longitudinal drive section which drives the optical component in a longitudinal direction of a longitudinal axis of the sheath in the sheath, the sheath comprising a plurality of balloons spaced at predetermined intervals on a part on the distal end portion side of an outer periphery and capable of inflating/deflating in a radial direction orthogonal to the longitudinal axis, a suction inlet located between the plurality of balloons to draw the body tissue at the part on the distal end portion side of the outer periphery by suction, a balloon inflation port connected to the balloons, and a suction port connected to the suction inlet, comprising a locking step of airtightly locking the plurality of balloons to an body cavity inner wall by supplying a fluid or a gas through the balloon inflation port to pressurize and inflate the balloons and an adhesion step of reducing pressure in a space formed between the plurality of locked balloons and the body cavity inner wall through the suction port to cause the body tissue to adhere strongly to the part on the distal end portion side of the outer periphery by suction.

As in the case of a drive control method for an optical probe according to a 13th aspect of the present invention, the drive control method for the optical probe according to the 12th aspect preferably further comprises a fluid supply control step of detecting internal pressure of each of the balloons and controlling supply of the fluid through the balloon inflation port in order to keep the balloon airtightly locked to the body cavity inner wall.

An endoscope apparatus according to a 14th aspect of the present invention comprises an optical probe according to any one of the first to 11th aspects and an endoscope having a treatment tool channel which is inserted through the sheath of the optical probe.

As has been described above, the present invention has the advantage of the capability to stably lock an OCT probe in a body cavity and acquire a blur-free tomographic image with high resolution.

### BRIEF DESCRIPTION OF THE DRAWINGS

FIG. 1 is a block diagram showing the internal configurations of an OCT probe and an OCT processor according to an embodiment of the present invention;

FIG. 2 is a cross-sectional view showing the configuration of an optical rotary joint to which a rotating-side optical fiber in FIG. 1 is connected;

FIG. 3 is a longitudinal cross-sectional view showing the configuration of the OCT probe in FIG. 1;

FIG. 4 is a cross-sectional view showing a cross section taken along line A-A in FIG. 3;

FIG. 5 is a cross-sectional view showing a cross section taken along line B-B in FIG. 3;

FIG. 6 is a cross-sectional view showing a cross section taken along line C-C in FIG. 3;

FIG. 7 is a cross-sectional view showing a cross section taken along line D-D in FIG. 3;

FIG. 8 is a cross-sectional view showing a cross section taken along line E-E in FIG. 3;

FIG. 9 is a view showing the configuration of each balloon in FIG. 3;

FIG. 10 is a flow chart for explaining the operation of the OCT processor in relation to the OCT probe in FIG. 3;

FIG. 11 is a schematic view of a case where the interior of a body cavity is observed by the OCT probe in the process in FIG. 10;

FIG. 12 is a longitudinal cross-sectional view showing the configuration of a modification of the OCT probe in FIG. 1;

FIG. 13 is a view showing a diagnostic imaging apparatus used in combination with an endoscope apparatus, to which the OCT probe in FIG. 1 can be applied; and

FIG. 14 is a view showing how a conventional OCT probe is inserted in a body cavity.

### DETAILED DESCRIPTION OF THE PREFERRED EMBODIMENTS

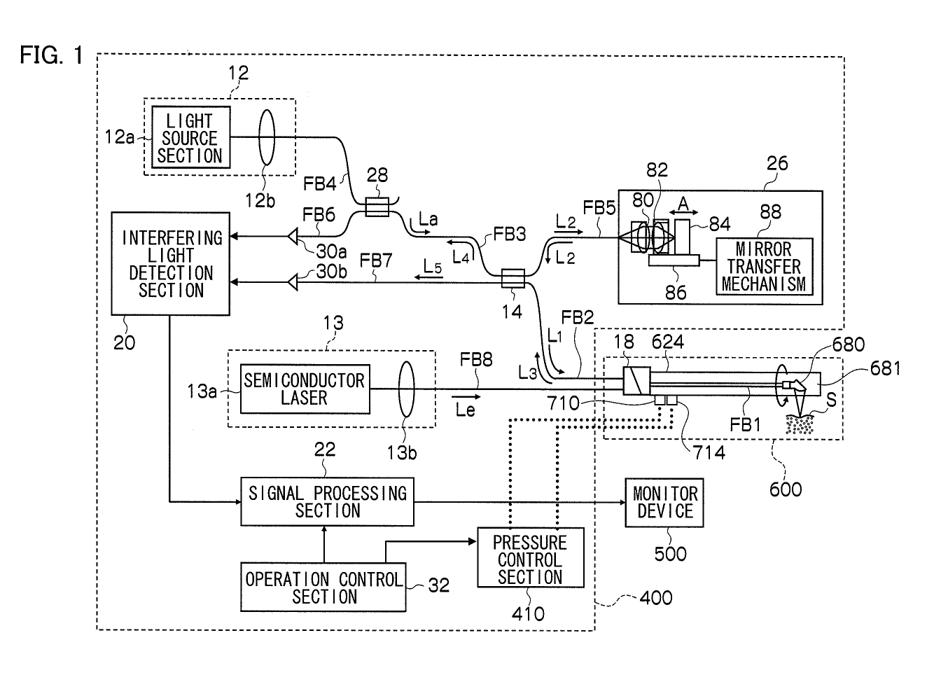

An embodiment of the present invention will be described in detail below with reference to the accompanying drawings. FIG. 1 is a block diagram showing the internal configurations of an OCT probe and an OCT processor according to the embodiment of the present invention.

As shown in FIG. 1, an OCT probe 600 and an OCT processor 400 according to this embodiment are intended to acquire an optical tomographic image of an object to be measured by an optical coherence tomography (OCT) measurement method.

The OCT processor 400 includes a first light source (first light source unit) 12 which emits a light beam La for measurement, an optical fiber coupler (demultiplexing and multiplexing section) 14 which demultiplexes the light beam La emitted from the first light source 12 into a measuring light beam (first rays of light) L1 and a reference light beam L2 and multiplexes a return light beam L3 from an object S to be measured serving as a test object and the reference light beam L2 to produce interfering light beams L4 and L5, the OCT probe 600 comprising a rotating-side optical fiber FB1 which guides the measuring light beam L1 obtained through demultiplexing by the optical fiber coupler 14 to the object to be measured and guides the return light beam L3 from the object to be measured, a fixed-side optical fiber FB2 which guides the measuring light beam L1 to the rotating-side optical fiber FB1 and guides the return beam L3 guided by the rotating-side optical fiber FB1, an optical connector 18 which rotatably connects the rotating-side optical fiber FB1 to the fixed-side optical fiber FB2 and transmits the measuring light beam L1 and the return light beam L3, an interfering light detection section 20 which detects the interfering light beams L4 and L5 produced by the optical fiber coupler 14 as interference signals, and a processing section 22 which processes the interference signals detected by the interfering light detection section 20 and acquires optical structure information. An image is displayed on a monitor device 500 on the basis of the optical structure information acquired by the processing section 22.

The OCT processor 400 also includes a second light source (second light source unit) 13 which emits an aiming light beam (second rays of light) Le for indicating a measurement mark, an optical path length adjustment section 26 which adjusts the optical path length of the reference light beam L2, an optical fiber coupler 28 which splits the light beam La emitted from the first light source 12, detection sections 30a and 30b which detect the interfering light beams L4 and L5 obtained through multiplexing by the optical fiber coupler 14, and an operation control section 32 which inputs various conditions to the processing section 22, changes settings, and performs other processes.

Note that various optical fibers FB including the rotating-side optical fiber FB1 and the fixed-side optical fiber FB2 (FB3, FB4, FB5, FB6, FB7, FB8, and the like) are used as light paths for guiding and transmitting various light beams including the emitted light beam La, the aiming light beam Le, the measuring light beam L1, the reference light beam L2, and the return light beam L3 described above between components such as the optical devices in the OCT processor 400 shown in FIG. 1.

The first light source 12 emits light for OCT measurement (e.g., laser light with a wavelength of 1.3 μm or low-coherence light). The first light source 12 is a light source which emits the laser light beam La with, e.g., a center wavelength of 1.3 μm in the infrared region while periodically sweeping the frequency of the light beam. The first light source 12 includes a light source 12a which emits the laser light beam or low-coherence light beam La and a lens 12b which focuses the light beam La emitted from the light source 12a. Although the details will be described later, the light beam La emitted from the first light source 12 passes through the optical fibers FB4 and FB3 and is split into the measuring light beam L1 and the reference light beam L2 by the optical fiber coupler 14, and the measuring light beam L1 is inputted to the optical connector 18.

The second light source 13 emits visible light as the aiming light beam Le for facilitating confirmation of a part to be measured. For example, red semiconductor laser light with a wavelength of 0.66 μm, He—Ne laser light with a wavelength of 0.63 μm, blue semiconductor laser light with a wavelength of 0.405 μm, or the like can be used as the aiming light beam Le. The second light source 13 includes a semiconductor laser 13a which emits, e.g., red, blue, or green laser light and a lens 13b which focuses the aiming light beam Le emitted from the semiconductor laser 13a. The aiming light beam Le emitted from the second light source 13 is inputted to the optical connector 18 through the optical fiber FB8.

At the optical connector 18, the measuring light beam L1 and the aiming light beam Le are multiplexed, and a resultant light beam is guided to the rotating-side optical fiber FB1 in the OCT probe 600.

The optical fiber coupler (demultiplexing and multiplexing section) 14 is composed of, e.g., a 2×2 optical fiber coupler and is optically connected to the fixed-side optical fiber FB2, the optical fiber FB3, the optical fiber FB5, and the optical fiber FB7.

The optical fiber coupler 14 splits the light beam La incident from the first light source 12 through the optical fibers FB4 and FB3 into the measuring light beam (first rays of light) L1 and the reference light beam L2 and causes the measuring light beam L1 to enter the fixed-side optical fiber FB2 and the reference light beam L2 to enter the optical fiber FB5.

The optical fiber coupler 14 also multiplexes the light beam L2, which is inputted to the optical fiber FB5, is frequency shifted and changed in optical path length by the optical path length adjustment section 26 (to be described later), and is returned through the optical fiber FB5, and the light beam L3, which is acquired by the OCT probe 600 and is guided from the fixed-side optical fiber FB2 and emits a resultant light beam to the optical fiber FB3 (FB6) and the optical fiber FB7.

The OCT probe 600 is connected to the fixed-side optical fiber FB2 through the optical connector 18. The measuring light beam L1 from the fixed-side optical fiber FB2 is multiplexed with the aiming light beam Le and is inputted to the rotating-side optical fiber FB1 through the optical connector 18. The inputted measuring light beam L1 multiplexed with the aiming light beam Le is transmitted by the rotating-side optical fiber FB1 and is applied to the object S to be measured. The return light beam L3 from the object S to be measured is acquired, and the acquired return light beam L3 is transmitted by the rotating-side optical fiber FB1 and is emitted to the fixed-side optical fiber FB2 through the optical connector 18.

The optical connector 18 multiplexes the measuring light beam (first rays of light) L1 and the aiming light beam (second rays of light) Le.

The interfering light detection section 20 is connected to the optical fiber FB6 and the optical fiber FB7 and detects the interfering light beams L4 and L5, which are produced by multiplexing the reference light beam L2 and the return light beam L3 in the optical fiber coupler 14, as interference signals.

The OCT processor 400 has the detector 30a, which is provided on the optical fiber FB6 diverging from the optical fiber coupler 28 and detects the light intensity of the interfering light beam L4, and the detector 30b on an optical path of the optical fiber FB7, which detects the light intensity of the interfering light beam L5.

The interfering light detection section 20 detects the intensity of reflected light (or backscattered light) at each depth position of the object S to be measured by Fourier-transforming the interfering light beam L4 detected from the optical fiber FB6 and the interfering light beam L5 detected from the optical fiber FB7 on the basis of detection results from the detectors 30a and 30b.

The processing section 22 acquires optical structure information from the interference signals extracted by the interfering light detection section 20. The processing section 22 generates an optical three-dimensional structure image on the basis of the acquired optical structure information and outputs an image obtained by subjecting the optical three-dimensional structure image to various processes to the monitor device 500.

The optical path length adjustment section 26 is arranged on the side where the reference light beam L2 is emitted from the optical fiber FB5 (i.e., at the end opposite to the optical fiber coupler 14 of the optical fiber FB5).

The optical path length adjustment section 26 includes a first optical lens 80 which collimates a light beam emitted from the optical fiber FB5, a second optical lens 82 which focuses the light beam collimated by the first optical lens 80, a reflecting mirror 84 which reflects the light beam focused by the second optical lens 82, a base 86 which supports the second optical lens 82 and the reflecting mirror 84, and a mirror transfer mechanism 88 which moves the base 86 in a direction parallel to an optical axis direction. The optical path length adjustment section 26 adjusts the optical path length of the reference light beam L2 by changing the distance between the first optical lens 80 and the second optical lens 82.

The first optical lens 80 collimates the reference light beam L2 emitted from a core of the optical fiber FB5 and focuses the reference light beam L2 reflected by the reflecting mirror 84 onto the core of the optical fiber FB5.

The second optical lens 82 focuses the reference light beam L2 collimated by the first optical lens 80 onto the reflecting mirror 84 and collimates the reference light beam L2 reflected by the reflecting mirror 84. As described above, the first optical lens 80 and the second optical lens 82 constitute a confocal optical system.

The reflecting mirror 84 is arranged at the focal point of a light beam focused by the second optical lens 82 and reflects the reference light beam L2 focused by the second optical lens 82.

With the above-described configuration, the reference light beam L2 emitted from the optical fiber FB5 is collimated by the first optical lens 80 and is focused onto the reflecting mirror 84 by the second optical lens 82. After that, the reference light beam L2 reflected by the reflecting mirror 84 is collimated by the second optical lens 82 and is focused onto the core of the optical fiber FB5 by the first optical lens 80.

The base 86 fixedly holds the second optical lens 82 and the reflecting mirror 84. The mirror transfer mechanism 88 moves the base 86 in an optical axis direction of the first optical lens 80 (a direction indicated by an arrow A in FIG. 1).

Movement of the base 86 in the direction indicated by the arrow A effected by the mirror transfer mechanism 88 changes the distance between the first optical lens 80 and the second optical lens 82. This allows adjustment of the optical path length of the reference light beam L2.

The operation control section 32 includes an input device such as a keyboard or a mouse and a control device which manages various conditions on the basis of entered information and is connected to the processing section 22. The operation control section 32 performs, e.g., inputting of various processing conditions and the like to the processing section 22 and setting and change of the processing conditions in the processing section 22 in accordance with an operator's instructions entered from the input device.

Note that the operation control section 32 may display an operation screen on the monitor device 500 or display an operation screen on a separately provided display section. The operation control section 32 may be configured to control the operation of the first light source 12, the second light source 13, the optical connector 18, the interfering light detection section 20, the optical path length adjustment section 26, and the detection sections 30a and 30b and set various conditions for the components.

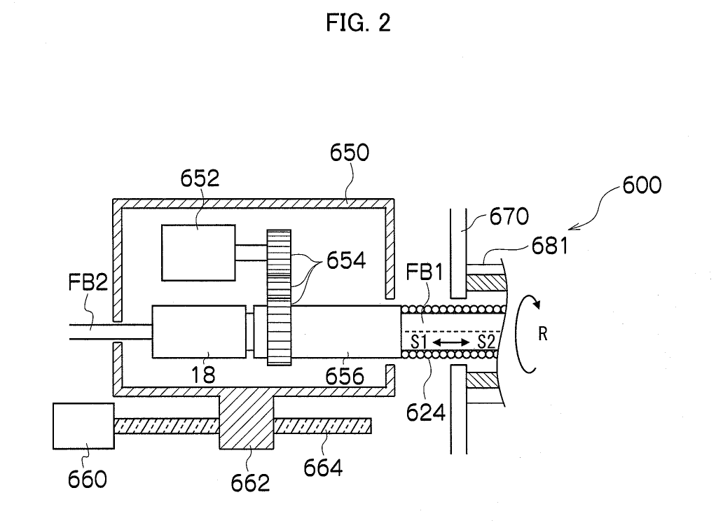

As shown in FIG. 2, the rotating-side optical fiber FB1 and the fixed-side optical fiber FB2 are connected by the optical connector 18. The optical fibers FB1 and FB2 are optically connected such that rotation of the rotating-side optical fiber FB1 is not transmitted to the fixed-side optical fiber FB2. The rotating-side optical fiber FB1 is arranged to be rotatable with respect to a sheath 681 and be movable in a longitudinal direction of the sheath 681.

A torque transmission coil 624 is fixed to an outer periphery of the rotating-side optical fiber FB1. The rotating-side optical fiber FB1 and the torque transmission coil 624 are connected to an optical rotary joint (not shown) in the optical connector 18.

In the OCT probe 600, the rotating-side optical fiber FB1, the torque transmission coil 624, and a ball lens 680 (see FIG. 1) as an optical component are configured to be movable in the sheath 681 both in a direction indicated by an arrow S1 (a direction toward a forceps outlet) and in a direction indicated by an arrow S2 (a direction toward a distal end of the sheath 681) by a forward and reverse drive section (to be described later) which is provided at the optical connector 18.

The sheath 681 is fixed to a fixed member 670. In contrast, the rotating-side optical fiber FB1 and the torque transmission coil 624 are connected to a rotary cylinder 656. The rotary cylinder 656 is configured to rotate in accordance with rotation of a motor 652 transmitted through a gear 654. The rotary cylinder 656 is connected to the optical rotary joint of the optical connector 18. The measuring light beam L1 and the return light beam L3 are transmitted between the rotating-side optical fiber FB1 and the fixed-side optical fiber FB2 through the optical connector 18.

A frame 650 incorporating the components includes a support member 662. The support member 662 has a tapped hole (not shown). The frame 650 occludes with a ball screw 664 for forward and reverse movement at the tapped hole (not shown) of the support member 662. The ball screw 664 for forward and reverse movement connects with a motor 660. The tapped hole, the ball screw 664 for forward and reverse movement, the motor 660, and the like constitute the forward and reverse drive section as a forward and reverse movement device.

The forward and reverse drive section for the optical rotary joint of the optical connector 18 moves the frame 650 back and forth by rotatable driving of the motor 660. With the forward and reverse movement, the forward and reverse drive section is capable of moving the rotating-side optical fiber FB1, the torque transmission coil 624, the fixed member 670, and the ball lens 680 in the directions S1 and S2 in FIG. 2.

Note that the motor 660 performs forward and reverse driving in predetermined steps (e.g., 1 mm steps). At each predetermined step, the motor 652 rotates the rotating-side optical fiber FB1, the torque transmission coil 624, and the ball lens 680 once, thereby applying the measuring light beam L1 to the object S to be measured for radial scanning.

In the OCT probe 600 with the above-described configuration, the rotating-side optical fiber FB1 and the torque transmission coil 624 are rotated in a direction indicated by an arrow R in FIG. 2 by the optical rotary joint of the optical connector 18. With the rotation, the OCT probe 600 applies the measuring light beam L1 emitted from the ball lens 680 to the object S to be measured while performing radial scanning in the direction indicated by the arrow R (a circumferential direction of the sheath 681) and acquires the return light beam L3.

For this reason, at each angle along the circumferential direction of the sheath 681, a desired part of the object S to be measured can be accurately captured, and the return light beam L3 reflected from the object S to be measured can be acquired.

When a plurality of pieces of optical structure information are to be acquired to generate an optical three-dimensional structure image, the ball lens 680 is first moved to an end of a movable range in the direction indicated by the arrow S1 in FIG. 2 by the forward and reverse drive section for the optical rotary joint of the optical connector 18. The ball lens 680 moves in the direction S2 in predetermined steps to the other end of the movable range while acquiring pieces of optical structure information composed of tomographic images or the ball lens 680 alternates between acquisition of optical structure information and movement in the direction S2 in FIG. 2 in predetermined steps until the ball lens 680 reaches the other end of the movable range.

As described above, the OCT probe 600 and the OCT processor 400 according to this embodiment are capable of acquiring a plurality of pieces of optical structure information for a desired range of the object S to be measured and acquiring an optical three-dimensional structure image on the basis of the plurality of acquired pieces of optical structure information.

More specifically, the OCT probe 600 and the OCT processor 400 acquire a piece of optical structure information in a depth direction of the object S to be measured (a first direction) from interference signals. The OCT probe 600 and the OCT processor 400 are capable of acquiring a piece of optical structure information on a scan plane formed by the depth direction of the object S to be measured (the first direction) and a direction almost perpendicular to the depth direction (a second direction) by radial scanning of the object S to be measured in the direction indicated by the arrow R in FIG. 2 (the circumferential direction of the sheath 681) and are further capable of acquiring a plurality of pieces of optical structure information for generating an optical three-dimensional structure image by moving the scan plane along a direction almost perpendicular to the scan plane (a third direction).

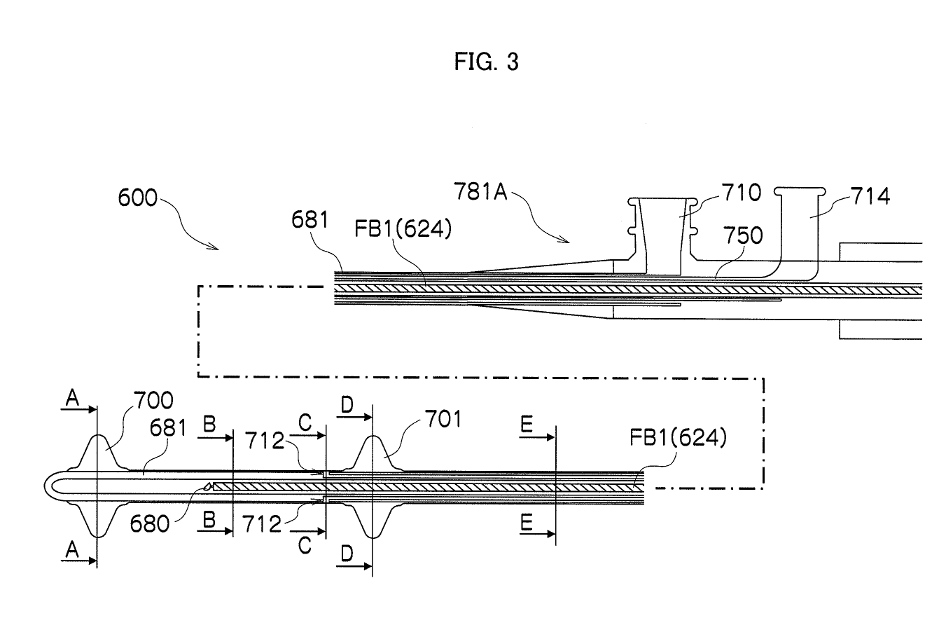

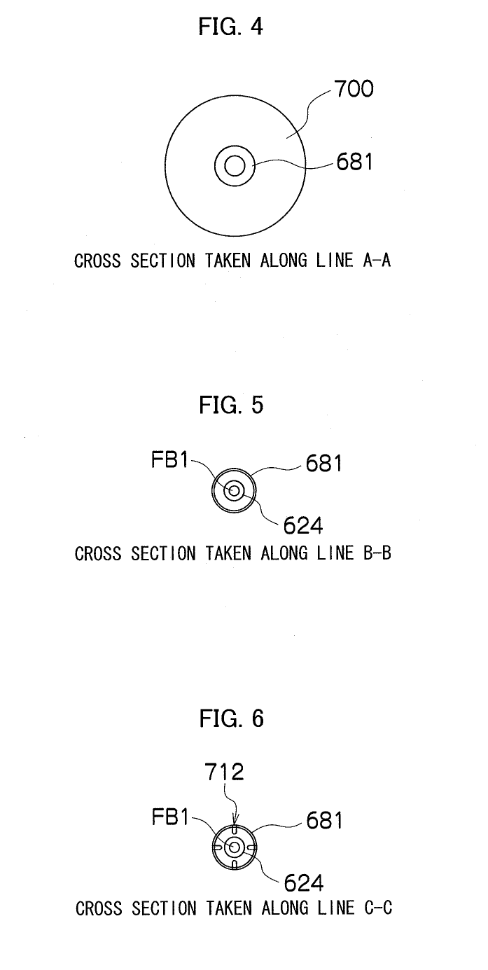

A diagnostic imaging apparatus according to the embodiment of the present invention will be described in detail below with reference to the drawings. FIG. 3 is a schematic cross-sectional view of the OCT probe in FIG. 1. FIGS. 4 to 8 are cross-sectional views showing cross sections at points (a cross section taken along line A-A, a cross section taken along line B-B, a cross section taken along line C-C, a cross section taken along line D-D, and a cross section taken along line E-E) in FIG. 3. FIG. 9 is a view showing the configuration of each balloon in FIG. 3.

As described above, the OCT probe 600 performs radial scanning while rotating the ball lens 680 by rotating the torque transmission coil 624 arranged outside the rotating-side optical fiber FB1 with the ball lens 680 at its distal end. At the same time, the OCT probe 600 performs longitudinal scanning by means of the forward and reverse drive section for the optical rotary joint of the optical connector 18. This allows spiral scanning. A combination of the rotating-side optical fiber FB1, the torque transmission coil 624, and the ball lens 680 will be referred to as an image core hereinafter.

The sheath 681 incorporates the extending image core and includes two cylindrical balloons 700 and 701 which are respectively arranged before and behind a longitudinal scan range at a distal end portion. The balloons 700 and 701 are connected to a balloon inflation port 710 which is provided at a proximal section 681A of the sheath 681. Although not shown, saline, an X-ray contrast medium, or gas such as air or carbon dioxide is injected into the balloons 700 and 701 by a pressure device (not shown) such as a syringe with lock or an indeflator through the balloon inflation port 710 to increase or reduce the pressures in the balloons 700 and 701. In the sheath 681, the balloons 700 and 701 can be inflated and deflated by increasing and reducing the pressures in the balloons 700 and 701 under control of a pressure control section 410 (see FIG. 1) in the OCT processor 400. Note that the X-ray contrast medium may be used in undiluted form or may be diluted with saline.

The balloons 700 and 701 are made of a flexible material such as silicone rubber and are configured to closely fit microscopic asperities on the surface of a living body when they are inflated. Although silicone rubber is used here, the present invention is not limited to the material. Any other material such as latex rubber or nylon may be used as long as the material meets the requirement.

As will be described later, the OCT probe 600 is used while a space between the two balloons 700 and 701 is under negative pressure, after the balloons 700 and 701 are inflated. In order to reduce deformation of the balloons 700 and 701 attracted to each other in this case, two end portions of each of the balloons 700 and 701 are formed as thick-wall portions 720, and a central portion is formed as a thin-wall portion 721, as shown in FIG. 9. It is desirable that the flexibility of each of the balloons 700 and 701 is enhanced at the thin-wall portion 721 to improve the ability to closely fit a luminal tissue in a radial direction with respect to a rotation axis of the OCT probe 600 and that the flexibility of the balloon 700 and 701 is lowered at the thick-wall portions 720 on both sides (in a longitudinal direction) to reduce deformation.

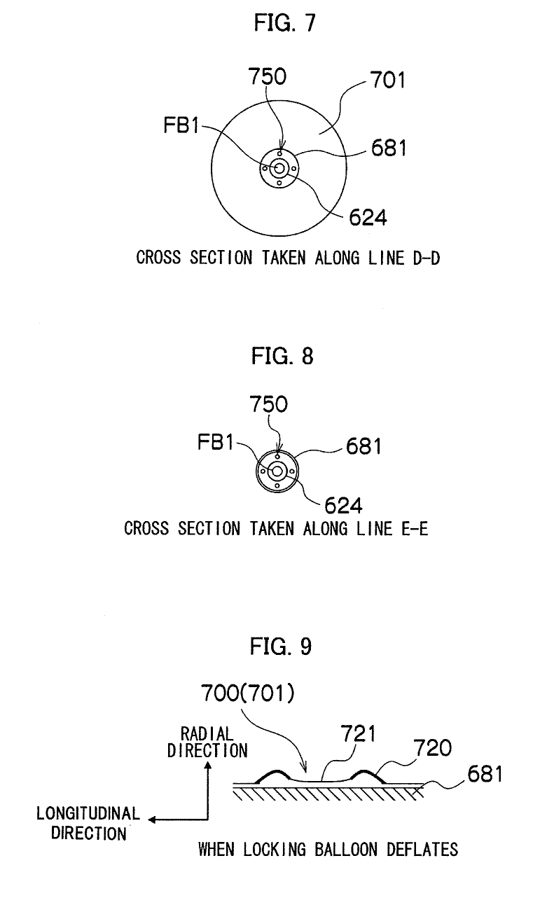

A plurality of suction inlets 712 are formed in the sheath 681 between the two balloons 700 and 701. Each suction inlet 712 is connected to a suction port 714 of the proximal section 681A through a communication channel 750 (see FIGS. 7 and 8). In the sheath 681, connection of a vacuum pump (not shown) to the suction port 714 allows suction through the suction inlets 712 under control of the pressure control section 410 (see FIG. 1) in the OCT processor 400.

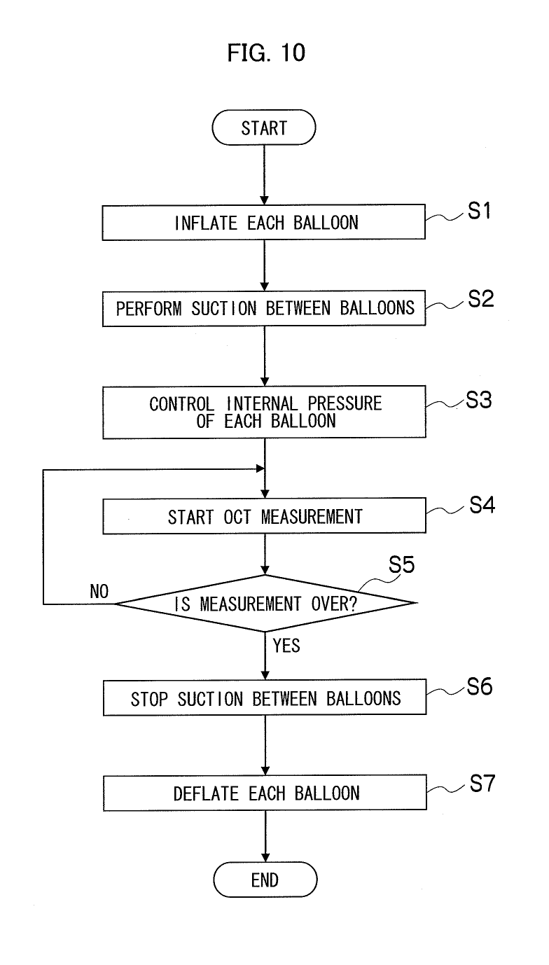

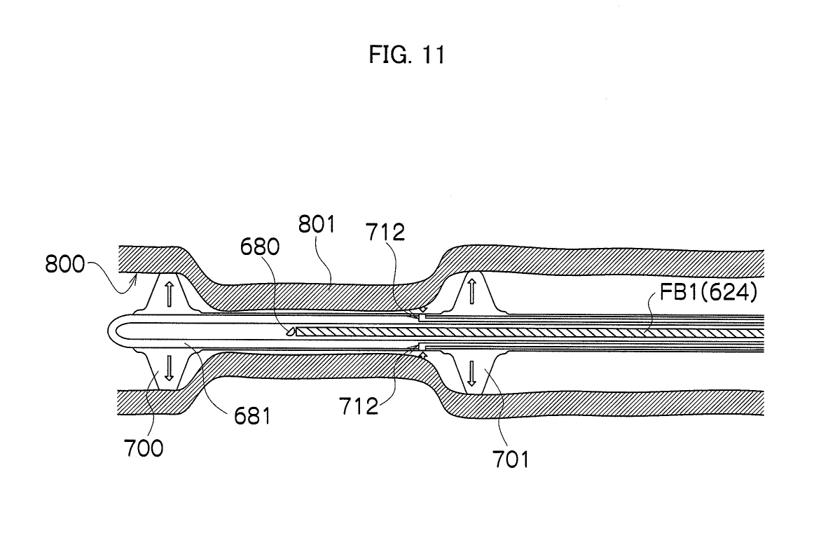

The procedure from when the OCT probe 600 is inserted into an affected part to when the OCT processor 400 acquires a tomographic image will be described below using the flow chart in FIG. 10 with reference to FIG. 11. FIG. 10 is a flow chart for explaining the operation of the OCT processor in relation to the OCT probe. FIG. 11 is a schematic view of a case where the interior of a body cavity is observed by the OCT probe in the process in FIG. 10.

When the OCT probe 600 is to be inserted into a body cavity, it is for example inserted into a coelomic tissue of a bile duct, a pancreatic duct, the large intestine, or the like through a forceps outlet of an endoscope (not shown) and is advanced to a lesioned part, as in the case of a common OCT probe.

As shown in FIG. 10, when the distal end of the sheath 681 of the OCT probe 600 reaches a part to be examined (observed), the OCT processor 400 injects saline or an X-ray contrast medium into the balloons 700 and 701 through the balloon inflation port 710 by the pressure device (not shown) and pressurizes the balloons 700 and 701 under control of the pressure control section 410. With the injection of the saline or the X-ray contrast medium, the balloons 700 and 701 inflate, and the OCT probe 600 is airtightly locked and fixed to a body cavity inner wall 800 through the balloons 700 and 701 (step S1). At this time, the body cavity is prevented from inflating excessively due to the inflation of the balloons 700 and 701.

In a part observable by endoscopy, such as the large intestine, a fluid such as saline may be injected into the balloons 700 and 701. If the OCT probe 600 is inserted into a part unobservable by endoscopy, such as a bile duct or a pancreatic duct, observation is performed under fluoroscopy. Where and how the OCT probe 600 is inserted and the shapes of the balloons 700 and 701 can be observed by injecting an X-ray contrast medium into the balloons 700 and 701.

The OCT processor 400 performs depressurization through the suction port 714 (see FIG. 3) of the proximal section 681A by means of the vacuum pump (not shown) under control of the pressure control section 410. With the depressurization, the OCT processor 400 draws a body tissue 801 at the part to be observed through the suction inlets 712 at the distal end portion of the sheath 681 by suction such that the body tissue 801 adheres strongly to the OCT probe 600 (step S2).

The OCT processor 400 monitors the balloons 700 and 701 by means of the pressure control section 410. When the body tissue 801 is drawn through the suction inlets 712 by suction such that the body tissue 801 adheres strongly to the OCT probe 600, an enclosed space formed between the balloons 700 and 701 and the body cavity inner wall 800 is brought under negative pressure. In this state, the balloons 700 and 701 are deformed toward the enclosed space, and the internal pressures of the balloons 700 and 701 decrease. This may reduce the airtightness between the balloons 700 and 701 and the body cavity inner wall 800. Accordingly, the OCT processor 400 controls the internal pressures of the balloons 700 and 701 to a predetermined pressure by the pressure control section 410 (step S3).

In this state, the OCT processor 400 simultaneously performs radial scanning by rotating a drive shaft and performs longitudinal scanning at a constant rate. The combination allows spiral scanning. The OCT processor 400 starts OCT measurement (step S4). Since the OCT processor 400 acquires three-dimensional tomographic image data of the body cavity in this situation, a blur-free image can be acquired with high resolution.

The OCT processor 400 determines the presence or absence of instructions to end the OCT measurement (step S5). If instructions to end the OCT measurement are issued, the process shifts to step S6. Otherwise, the process returns to step S4.

The OCT processor 400 stops suction in the space formed between the balloons 700 and 701 and the body cavity inner wall 800 through the suction inlets 712 under control of the pressure control section 410 and causes the body tissue 801 to lose its adhesion to the OCT probe 600 in step S6. The OCT processor 400 sucks the saline or the X-ray contrast medium from the balloons 700 and 701 under control of the pressure control section 410 to deflate the balloons 700 and 701 in step S7 and ends the process.

As described above, in this embodiment, a plurality of balloons (e.g., the two balloons 700 and 701) communicating with the balloon inflation port 710 in the longitudinal direction are provided at a distal end portion of the OCT probe 600, and the suction inlets 712 communicating with the suction port 714 are provided between the balloons 700 and 701. The OCT processor 400 controls the pressure device (not shown) connected to the balloon inflation port 710 and the vacuum pump (not shown) connected to the suction port 714. With this control, the distal end portion of the OCT probe 600 can be stably locked to the body cavity inner wall 800, and the body tissue 801 between the balloons 700 and 701 can be brought into contact with an outer peripheral surface of the sheath 681 of the OCT probe 600. The OCT processor 400 is thus capable of acquiring a blur-free tomographic image with high resolution.

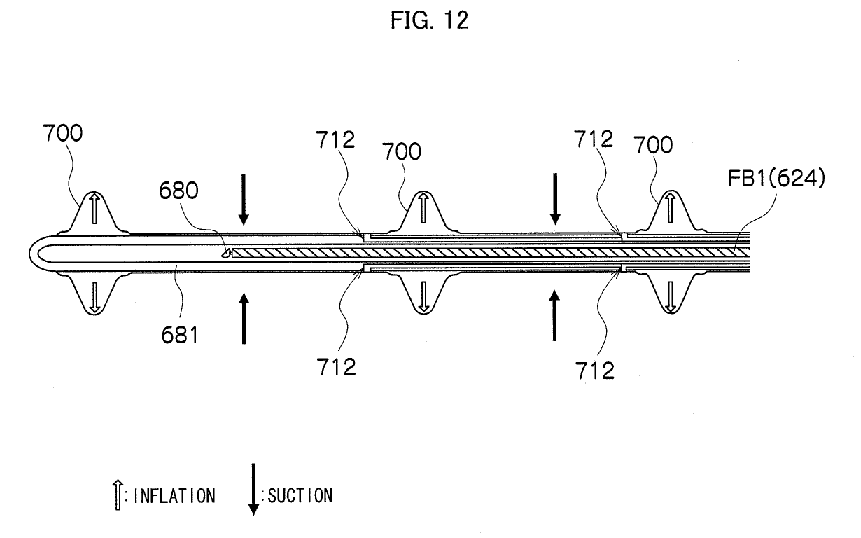

Note that although this embodiment has described a case where two balloons are provided at the distal end portion of the OCT probe 600, the present invention is not limited to this. For example, the configuration may be such that three balloons, the balloons 700 and 701 and a balloon 702 are provided along a longitudinal axis of the sheath 681, and the suction inlet 712 is provided between the balloons 700 and 701 and between the balloon 701 and 702, as shown in FIG. 12. It should be appreciated that the number of balloons may be three or more.

The OCT probe 600 according to this embodiment can be applied to a diagnostic imaging apparatus used in combination with an endoscope apparatus.

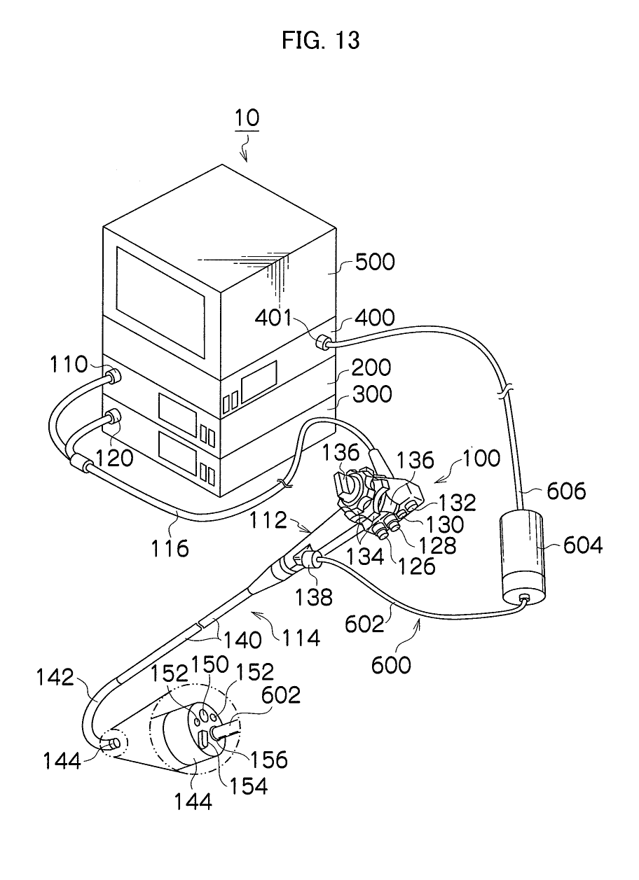

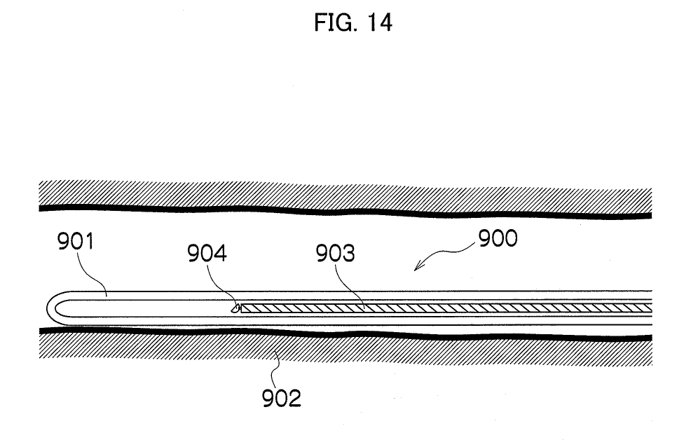

More specifically, as shown in FIG. 13, a diagnostic imaging apparatus 10 used in combination with the OCT probe 600 according to this embodiment and an endoscope apparatus is mainly composed of an endoscope 100, an endoscope processor 200, a light source device 300, the OCT processor 400 as a living body tomographic image generation device, and the image display section 500 that is a monitor device as a display device. Note that the endoscope processor 200 may be configured to incorporate the light source device 300.

The endoscope 100 includes a proximal operation section 112 and an insertion section 114 which is provided to be continuous with the proximal operation section 112. An operator holds and operates the proximal operation section 112 and performs observation by inserting the insertion section 114 into a body of an examinee.

A forceps insertion section 138 is provided at the proximal operation section 112 and communicates with a forceps outlet 156 of a distal end portion 144 through a forceps channel (not shown) provided in the insertion section 114. In the diagnostic imaging apparatus 10, the OCT probe 600 as a probe is inserted through the forceps insertion section 138 and is led out through the forceps outlet 156. The OCT probe 600 is composed of an insertion section 602 which is inserted through the forceps insertion section 138 and is lead out through the forceps outlet 156, an operation section 604 which is intended for an operator to operate the OCT probe 600, and a cable 606 which is connected to the OCT processor 400 through a connector 401.

An observation optical system 150, an illumination optical system 152, and a CCD (not shown) are disposed at the distal end portion 144 of the endoscope 100.

The observation optical system 150 forms an image of a test object on a light-receiving surface of the CCD (not shown), and the CCD converts the image of the test object into electric signals by means of light-receiving elements. The CCD according to this embodiment is a color CCD in which color filters of the three primary colors (red (R), green (G), and blue (B)) are arranged in a predetermined pattern (a Bayer pattern or a honeycomb pattern) to correspond to pixels of the CCD.

The light source device 300 causes visible light to enter a light guide (not shown). One end of the light guide is connected to the light source device 300 through an LG connector 120, and the other end of the light guide faces the illumination optical system 152. A light beam emitted from the light source device 300 passes through the light guide and is emitted from the illumination optical system 152 to illuminate a visual field range of the observation optical system 150.

An image signal outputted from the CCD is inputted to the endoscope processor 200 through an electric connector 110. The analog image signal is converted into a digital image signal in the endoscope processor 200 and is subjected to processing required for display on a screen of the monitor device 500.

As described above, data of an observation image acquired by the endoscope 100 is outputted to the endoscope processor 200, and the image is displayed on the monitor device 500 connected to the endoscope processor 200.

An optical probe, a drive control method for the optical probe, and an endoscope apparatus according to the present invention have been described in detail above. The present invention, however, is not limited to the above-described example. Of course, various improvements and modifications may be made without departing from the scope of the present invention.

What is claimed is:

1. An optical probe, comprising:
a sheath to be inserted into a body cavity;
an optical fiber inside the sheath;
an optical component attached to a distal end portion of the optical fiber inside the sheath;
wherein a light beam transmitted through the optical fiber is emitted from the optical component toward a body tissue in the body cavity, and
wherein the sheath comprises
a plurality of balloons spaced at predetermined intervals on a part on the distal end portion side of an outer periphery and capable of inflating/deflating in a radial direction orthogonal to a longitudinal axis,
a suction inlet located between the plurality of balloons to draw the body tissue at the part on the distal end portion side of the outer periphery by suction,
a balloon inflation port connected to the balloons, and
a suction port connected to the suction inlet, and
the plurality of balloons are airtightly locked to an body cavity inner wall by supplying a fluid or a gas through the balloon inflation port to pressurize and inflate the balloons, and pressure in a space formed between the plurality of locked balloons and the body cavity inner wall is reduced through the suction port to cause the body tissue to adhere strongly to the part on the distal end portion side of the outer periphery by suction.

2. The optical probe according to claim 1, wherein the balloon inflation port and the suction port are provided at a proximal end portion of the sheath.

3. The optical probe according to claim 1, wherein the optical fiber is arranged in a drive shaft which rotatably drives the optical fiber, and radial scanning is performed in the body cavity by rotatably driving the optical component.

4. The optical probe according to claim 3, wherein the drive shaft is also movable in a longitudinal direction, and spiral scanning is performed in the body cavity by rotatably driving the optical component and driving the optical component back and forth within a longitudinal driving range.

5. The optical probe according to claim 1, wherein the optical component comprises a ball lens having a reflecting surface which bends a traveling direction of the light beam transmitted through the optical fiber almost at a right angle.

6. The optical probe according to claim 1, wherein the optical fiber transmits wavelength swept laser light into the body cavity.

7. The optical probe according to claim 1, wherein the balloons are each thicker at two end portions than at a central portion.

8. The optical probe according to claim 1, further comprising: a fluid supply control device which detects internal pressure of each of the balloons and controls supply of the fluid through the balloon inflation port in order to keep the balloon airtightly locked to the body cavity inner wall.

9. The optical probe according to claim 1, wherein the fluid is one of an X-ray contrast medium and a fluid containing an X-ray contrast medium.

10. The optical probe according to claim 1, wherein the fluid is saline.

11. The optical probe according to claim 1, wherein the optical fiber transmits wavelength swept laser light into the body cavity.

12. A drive control method for an optical probe which comprises an optical fiber and an optical component attached to a distal end portion of the optical fiber inside a sheath to be inserted into a body cavity and emits a light beam transmitted through the optical fiber from the optical component toward a body tissue in the body cavity, the optical probe further comprising a longitudinal drive section which drives the optical component in a longitudinal direction of a longitudinal axis of the sheath in the sheath, the sheath comprising a plurality of balloons spaced at predetermined intervals on a part on the distal end portion side of an outer periphery and capable of inflating/deflating in a radial direction orthogonal to the longitudinal axis, a suction inlet located between the plurality of balloons to draw the body tissue at the part on the distal end portion side of the outer periphery by suction, a balloon inflation port connected to the balloons, and a suction port connected to the suction inlet, comprising the steps of:
a locking step of airtightly locking the plurality of balloons to an body cavity inner wall by supplying a fluid or a gas through the balloon inflation port to pressurize and inflate the balloons, and
an adhesion step of reducing pressure in a space formed between the plurality of locked balloons and the body cavity inner wall through the suction port to cause the body tissue to adhere strongly to the part on the distal end portion side of the outer periphery by suction.

13. The drive control method for the optical probe according to claim 12, further comprising: a fluid supply control step of detecting internal pressure of each of the balloons and controls supply of a fluid through the balloon inflation port in order to keep the balloon airtightly locked to the body cavity inner wall.

14. An endoscope apparatus comprising an optical probe according to claim 1 and an endoscope having a treatment tool channel which is inserted through the sheath of the optical probe.
